(19) United States
(12) Patent Application Publication
(10) Pub. No.: US 2026/0275054 A1
(43) Pub. Date: Sep. 17, 2026

(54) STORAGE GEL

(71) Applicants: UNIVERSITY OF WARWICK, Coventry, Warwickshire (GB); THE UNIVERSITY COURT OF THE UNIVERSITY OF GLASGOW, Glasgow, Strathclyde (GB)

(72) Inventors: Matthew Ian Gibson, Coventry (GB); Muhammad Hasan, Coventry (GB); Sarah-Jane Richards, Coventry (GB); Ashfaq Ahmad, Coventry (GB); David John Adams, Glasgow (GB); Simona Bianco, Conversano (IT)

(21) Appl. No.: 19/516,356

(22) PCT Filed: Sep. 6, 2024
(86) PCT No.: PCT/GB2024/052335

§ 371 Date: Mar. 4, 2026

(30) Foreign Application Priority Data
Sep. 8, 2023 (GB) 2313756.5

(51) Int. Cl. C08J 3/075; A61K 9/06; A61K 47/30; C08G 83/00

(52) U.S. Cl. CPC C08J 3/075; A61K 9/06; A61K 47/30; C08G 83/008

(57) ABSTRACT

The present invention relates to a gel composition comprising: (a) a supramolecular gel formed from or comprising a network of one or more low molecular weight gelators (LMWGs) in an aqueous solution; wherein the supramolecular gel: (i) has a storage modulus (G′) of from 100 to 1,000,000 Pa; (ii) has a loss modulus (G″) such that G″/G′ is from 0.01 to 0.5; and (iii) has a breakage strain of from 0.1 to 300%. Additionally, the invention relates to processes for preparing such gel compositions. The present invention also relates to a gel composition comprising an encapsulated component, processes for preparing such gel compositions and processes relating to storing, transporting and extracting such encapsulated components.

The present invention relates to a gel composition comprising a supramolecular gel formed from or comprising a network of one or more low molecular weight gelators (LMWGs) in an aqueous solution. The composition is suitable for the storage and transport of therapeutic agents without the need for refrigeration. The invention also provides a gel composition additionally comprising a therapeutic agent encapsulated therein and a syringe comprising a gel composition of the invention. The invention also provides processes for preparing supramolecular gels, processes for encapsulating therapeutic agents within gels and for extracting such agents from gels, and methods for storing and transporting therapeutic agents encapsulated with supramolecular gels.

Advanced biologic therapies are distributed cold, using significant energy, limiting equitable distribution in low-resource countries, and placing responsibility on the user for correct storage and handling.

A long-standing challenge is how to formulate proteins and vaccines to retain function during storage and transport and to remove the burdens of cold-chain management. Any solution must be practical to use, with the protein or vaccine being released/applied using clinically-relevant triggers. Cold-chain management is the current best solution for protein transport, but it requires significant infrastructure and energy. For example, in research labs, a single freezer at −80° C. consumes as much energy per day as a small household.² This is especially challenging in low or middle income (LMIC) countries where the energy requirements exclude equitable sharing of advanced medicines. 75% of biological (i.e. protein/cell) therapies and all vaccines require cold-chain management; the cost of cold-chain management in clinical trials has increased by ~20% since 2015, reflecting this complexity. Bespoke formulations and excipients are currently required, with trehalose,³ sucrose or polymers⁴ widely used which stabilize proteins by replacing surface water molecules making denaturation thermodynamically less likely; this has enabled both freeze-dried proteins and frozen proteins. For example, the HPV (human papilloma virus) vaccine requires aluminium salt adjuvants to function, but these render it unstable against freeze/thaw,⁵ leading to a very complex and expensive supply chain. Furthermore, mRNA vaccines must be transported and stored at −80° C., limiting their geographical distribution. In short, protein stabilization is major challenge with no universal solution.^{6,7}

Consideration of the pathways of thermal protein deactivation shows that irreversible aggregation, rather than chemical degradation or unfolding, is the primary mechanism for loss of activity. For example, simple mechanical stimulation (shaking) of insulin leads to aggregation into amyloid-type fibers and loss of efficacy and bioavailability. For example, the gene therapy Zolgensma™ has just a 14-day shelf life, cannot be agitated, and must be retained between 2 and 8° C. Clearly, this is a barrier to wider use. Additionally, aggregation of recombinant human interferon beta (rhIFN-β) leads to immunogenicity for patients with multiple sclerosis.⁸ Emerging antibody-based therapies also have major problems with aggregation at all stages of their life cycles, necessitating complex formulation processes.

Maynard et al. have developed synthetic polymers with trehalose side chains for protein stabilization, enabling both freeze-drying and heating stabilization, but the conjugation reduces activity.⁹ However, for many therapeutic applications, covalent conjugation of a polymer using multistep synthetic chemistry and purification is not practical and has the requirement to seek regulatory approvals for each material, significantly increasing the barrier to clinical translation. This is a broad issue that means that any stored protein is eventually delivered mixed with its stabilization agents, rather than the desirable solution of protein in buffer.

Fully-reversible hydrogels based on PEG (polyethylene glycol) have emerged to protect diverse proteins against thermal stress^{10,11} and mRNA formulations have been stabilized in a dissolvable matrix.¹² Similarly, formulations which are frozen to prevent ice crystal growth suppress protein aggregation.¹³ However, in each case these gels are based upon new chemical entities (and hence a significant barrier to human use); may require a chemical stimulus (e.g. pH, sugars) to release the protein, which in practice must be dosed by the user; and take more than 1 hour to release the protein. Whilst potent, these strategies result in protein mixed with the gel-forming components, which require separate approval and rigorous evaluation of their safety profiles before being suitable for human use. Even materials based on PEG have immunological concerns for frequent exposure.^{11,14,15}

An ideal biologic storage/transport solution would: i) remove/reduce cold chain requirements; ii) be broadly applicable; iii) have zero or minimal excipients in the delivered protein solution; iv) not require complex chemical triggers to release; and v) tolerate high loadings up to 100 mg/mL

Here we solve each of these points using a simple, low-resource strategy.

Gels can be formed by the self-assembly of low molecular weight gelators (LMWG).^{16,17} These are small molecules that self-assemble to give long fibrous structures that entangle to form a 3-dimensional network. These gels tend to be very stiff but break at low strain. This breaking at low strain has been widely described as a drawback of these systems.^{18,19} However, here we use this perceived failing as a unique benefit enabling a mechanical trigger for homogeneous protein delivery that is not possible with conventional gels.

We show herein that low molecular weight supramolecular gel networks can physically entrap proteins preventing irreversible aggregation, and that they retain function at temperatures as high as 50° C. for at least 4 weeks.

The supramolecular gel networks of the invention form a stiff hydrogel which stabilizes proteins against thermal denaturation, combined with the ability to deliver pure, excipient-free protein upon mechanical release from the network.

Advantageously, the protein or other therapeutic agent can be loaded within the gel network at up to 10 wt % without impacting the mechanism of release. This stabilization and release synergy provides a formulation strategy for the intact delivery of pure, non-aggregated homogenous protein or other encapsulated component.

The invention may be implemented using a wide variety of different gelators provided that supramolecular gels formed therefrom satisfy certain physical criteria as defined herein, including having storage and loss moduli, and breakage strains within certain ranges. Details are provided herein of a number of gelators which can be used in the context of the invention and how to test further potential gelators to see whether gels formed from such gelators fall within the physical criteria defined herein.

It is therefore an object of the invention to provide a new scalable means of storing and transporting biologically-important molecules without the need for cold-chain management, while ensuring their safe and effective subsequent use.

In one embodiment, the invention provides a gel composition comprising:
(a) a supramolecular gel formed from or comprising a network of one or more low molecular weight gelators (LMWGs) in an aqueous solution;
   wherein the supramolecular gel:
(i) has a storage modulus (G′) of from 100 to 1,000,000 Pa;
(ii) has a loss modulus (G″) such that G″/G′ is from 0.01 to 0.5; and
(iii) has a breakage strain of from 0.1 to 300%;
   and preferably wherein:
(iv) at least 90% of the LMWGs in the supramolecular gel are capable of being removed by filtration of the gel composition through a filter.

In another embodiment, the invention provides a gel composition of the invention, which additionally comprises:
(b) an encapsulated component, wherein the encapsulated component is encapsulated within the supramolecular gel.

Preferably, the encapsulated component comprises a biological macromolecule.

In another embodiment, the invention provides a syringe comprising a gel composition of the invention.

In another embodiment, the invention provides a process for preparing a supramolecular gel, the process comprising the steps:
(a) preparing a composition comprising one or more low molecular weight gelators (LMWGs) in an aqueous solution; and
(b) triggering the gelation of the LMWGs in the composition;
   under conditions that such that a supramolecular gel is formed from a network of the one or more LMWGs in the aqueous solution;
wherein the supramolecular gel:
(i) has a storage modulus (G′) of from 100 to 1,000,000 Pa;
(ii) has a loss modulus (G″) such that G″/G′ is from 0.01 to 0.5;
(iii) has a breakage strain of from 0.1 to 300%;
   and wherein:
(iv) at least 90% of the LMWGs in the supramolecular gel are capable of being removed by filtration of the gel composition through a filter.

The invention also encompasses supramolecular gels which are obtained or are obtainable by such a process.

The invention also provides a process for encapsulating a component in a gel, the process comprising the step:
(a) encapsulating the component in a supramolecular gel of the invention.

The invention also provides a method of storing a component, the method comprising the step:
(a) storing a gel composition of the invention wherein the component is encapsulated within the supramolecular gel at a temperature above 5° C. for a duration of at least 24 hours.

The invention also provides a method of transporting a component, the method comprising the step:
(a) transporting a gel composition of the invention wherein the component is encapsulated within the supramolecular gel from a first location to a second location.

The invention also provides a process of extracting an encapsulated component from a gel composition which comprises the encapsulated component, the process comprising the steps:
(a) forcing a gel composition of the invention which comprises the encapsulated component through a filter having a pore size such that all or substantially all of the LMWGs are retained within the filter; and
(b) collecting encapsulated component which passes through the filter.

In another embodiment, the invention provides a kit comprising:
(i) one or more low molecular weight gelators (LMWGs); and
(ii) a syringe,
   optionally together with instructions for use.

As used herein, the term “gel” or “supramolecular gel” refers to a three-dimensional network of entangled or cross-linked fibrous structures that is expanded throughout its volume by an aqueous solution. The supramolecular gels of the invention comprise or are formed from small molecules or gelators (e.g. low molecular weight gelators) that self-assemble to give long fibrous structures that, under appropriate conditions, entangle to form a three-dimensional network of fibres (i.e. the gel network).

The supramolecular gels of the invention assemble via non-covalent interactions, such as π-stacking, hydrogen-bonding, Van der Waals interactions or a combination thereof to form volume-filling 3D networks. The supramolecular gels are substantially or completely free of covalent cross-links between the gelators.

In some embodiments, the supramolecular gel of the invention is a hydrogel. As used herein, the term “hydrogel” refers to a gel structure that is expanded throughout its volume by a swelling agent, e.g. water or another aqueous-based solution.

The gel is formed from a network of one or more low molecular weight gelators (LMWGs). As used herein, the term “gelator” refers to a small molecule that can (self-)assemble into a gel. A gelator may also be considered as a monomeric sub-unit which is capable of forming a (self-)assembled fibrous network. Under appropriate conditions, such gelators may entangle one another to form a three-dimensional gel network.

The gelator is a low-molecular-weight gelator (LMWG). In some embodiments, the gelator has a molecular weight of less than 3,000 Da. For example, the gelator of use in the invention may have a molecular weight of between 100-3,000 Da, preferably 200-2000 Da, and more preferably 300-1000 Da. The molecular weight of the gelator can be determined based on its chemical structure.

In some embodiments, the gelators are ones which are capable of forming one dimensional structures such as fibres, tubes and/or helical tapes. In some embodiments, the gelators are ones which are capable of self-assembling into structures such as these in water. In some embodiments, the gelators are ones wherein the self-assembly into these structures leads to entanglement of the structures into a 3 dimensional network that is capable of immobilising the solvent and forming a gel.

In some embodiments, the chemical structure of the gelator comprises one or more naphthalene groups. In some embodiments, the chemical structure of the gelator comprises one or more COOH groups.

The invention may be implemented using a wide variety of different gelators provided that supramolecular gels formed therefrom satisfy the physical criteria as defined herein. Details are given below of a number of gelators which may be used in the context of the invention. The person of skill in the art would be able to test these potential gelators to see whether gels formed from such gelators fall within the physical criteria defined herein.

Numerous LWMGs are known in the art (e.g. see de Loos, et a. “Design and Application of Self-Assembled Low Molecular Weight Hydrogels” European Journal of Organic Chemistry, vol. 2005, issue 17, pages 3615-3631 (2005) Estroff and Hamilton, “Water Gelation by Small Organic Molecules”, Chem. Rev. 2004, 104, 3, 1201-1218.)

In some embodiments, the gel is a hydrogel and the gelator is selected from the group consisting of:
(a) Peptide-amphiphiles (for example, “Supramolecular Assembly of Peptide Amphiphiles”, Hendricks et al., Acc. Chem. Res. 2017, 50, 10, 2440-2448);
(b) Amphipathic peptides (for example Bowerman and Nilsson, “Review self-assembly of amphipathic β-sheet peptides: Insights and applications”; Peptide Science, vol. 98, issue 3 (2012), pages 169-184);
(c) Aromatic peptides (for example, Fleming and Ulijn, “Design of nanostructures based on aromatic peptide amphiphiles”, Chem. Soc. Rev. (2014), 43, 8150-8177;
(d) Bile acids (for example, Zhang et al., “Supramolecular hydrogelation with bile acid derivatives: structures, properties and applications”, J. Mater. Chem. B, (2016), 4, 7506-7520 (https://pubs.rsc.org/en/content/articlelanding/2016/tb/c6tb02270g);
(e) Nucleosides (for example, Godoy-Gallardo et al., “Nucleoside-Based Supramolecular Hydrogels: From Synthesis and Structural Properties to Biomedical and Tissue Engineering Applications”, CS Biomater. Sci. Eng. 2023, 9, 1, 40-61; (https://pubs.acs.org/doi/10.1021/acsbiomaterials.2c01051);
(f) Sorbitols (for example, Okesola et al., “1,3:2,4-Dibenzylidene-d-sorbitol (DBS) and its derivatives—efficient, versatile and industrially-relevant low-molecular-weight gelators with over 100 years of history and a bright future”, Soft Matter, 2015, 11, 4768-4787; and
(g) Dendrimers (for example, Kaga et al, “Dendrimers and Dendrons as Versatile Building Blocks for the Fabrication of Functional Hydrogels”, Molecules 2016, 21(4), 497.

In some embodiments, the gelator may be selected from the group consisting of the gelators in the following table:

| | |
|---|---|
| CD-005 | 3-{[(1S)-1-(Dodecylcarbamoyl)-2-methylpropyl]carbamoyl}propanoic |
| | acid |
| 2NapIF | 2-[[3-methyl-2-[(2-naphthalen-2-yloxyacetyl)amino]pentanoyl]amino]-3- |
| | phenylpropanoic acid |
| 1NapFF | 2-[[2-[(2-naphthalen-1-yloxyacetyl)amino]-3-phenylpropanoyl]amino]-3- |
| | phenylpropanoic acid |
| 2NapFV | 3-methyl-2-[[2-[(2-naphthalen-2-yloxyacetyl)amino]-3- |
| | phenylpropanoyl]amino]butanoic acid |
| 7MeO2NapIF | 2-[[2-[[2-(7-methoxynaphthalen-2-yl)oxyacetyl]amino]-3- |
| | methylpentanoyl]amino]-3-phenylpropanoic acid |
| 1Br2NapLFF | 2-[[2-[[2-[[2-(1-bromonaphthalen-2-yl)oxyacetyl]amino]-4- |
| | methylpentanoyl]amino]-3-phenylpropanoyl]amino]-3-phenylpropanoic acid |

The gel is formed from a network of one or more different LMWGs. For example, the gel may be formed from a network of 1, 2, 3, 4 or 5 different LMWGs. Preferably, the gel is formed from a network of the same (i.e. 1) LMWGs.

In some embodiments, the gelator is present in the supramolecular gel composition at a concentration of 10 wt % or less, or 5 wt % or less, and preferably 1 wt % or less.

For example, the gelator may be present in the supramolecular gel composition at a concentration of about 1 wt %, 1.5 wt %, 2 wt %, 2.5 wt %, 3 wt %, 3.5 wt %, 4 wt %, 4.5 wt %, 5 wt %, 5.5 wt %, 6 wt %, 6.5 wt %, 7 wt %, 7.5 wt %, 8 wt %, 8.5 wt %, 9 wt %, 9.5 wt % or 10 wt % preferably, at about 1 wt %.

The minimum gelation concentration of the gel is preferably greater than 0.01 wt %, more preferably between 0.1 wt % and 0.5 wt %, e.g. about 0.1, 0.3 or 0.5 wt %.

The supramolecular gel may be defined according to its storage modulus and its loss modulus.

The supramolecular gel has a storage modulus (G′) of from 100 to 1,000,000 Pa. As used herein, the term “storage modulus” (also known in the art as G′) is defined as a measure of the stored energy of a material, i.e. that representing the elastic portion. In other words, the storage modulus represents the elastic nature (energy storage) of a material. In some embodiments, the gels are stiff or rigid (i.e. the gels have a relatively high G′ value). Storage modulus (G′) is normally expressed within the art in units of Pascals (Pa). The supramolecular gel has a storage modulus (G′) of from 100 to 1,000,000 Pa, and preferably from 500 to 100,000 Pa. In some embodiments, the supramolecular gel has a storage modulus (G′) of from 500 to 1,000, 1,000 to 5,000, 5,000 to 50,000 or 50,000 to 100,000 Pa. The storage modulus of the supramolecular gel may be measured by any method known in the art, for example by rheology. Preferably, the storage modulus is measured by a rheometer, as described herein in the Materials and Methods.

The supramolecular gel has a loss modulus (G″) such that G″/G′ is from 0.01 to 0.5. As used herein, the term “loss modulus” (also known in the art as G″) is defined as a measure of the energy of a material, i.e. that representing the viscous portion. In other words, the loss modulus represents the viscous nature (energy loss) of a material.

Generally, the supramolecular gel will have a G′ that is around an order of magnitude greater than G″. Loss modulus (G″) is normally expressed within the art in units of Pascals (Pa). In some embodiments, the loss modulus is preferably 100 to 80,000 Pa.

The loss modulus of the supramolecular gel may be measured by any method known in the art, for example by rheology. Preferably, the loss modulus is measured by a rheometer, as described herein in the Materials and Methods.

The ratio of G″ to G′ (i.e. G″/G′) is known in the art as the “loss tangent” or tan 6. This provides a measure of how much the stress and strain are out of phase with one another. The ratio of G″ to G′ (i.e. G″/G′) in the supramolecular gel is from 0.01 to 0.5, preferably 0.01 to 0.2.

The supramolecular gel may be defined according to its breakage strain. The supramolecular gel has a breakage strain of from 0.1 to 300%. As used herein, the term “breakage strain” may be defined as the point where G′ is 90% of its value in the linear viscoelastic region (i.e. at a very low strain). The supramolecular gel has a breakage strain of from 0.1 to 300%, e.g. from 0.5 to 100%, and preferably from 1 to 10%. The breakage strain may be measured by a rheometer, as described herein in the Materials and Methods.

In some embodiments, the gel is essentially insoluble or is completely insoluble.

The supramolecular gels are stable and maintain their gel networks as described herein across a wide temperature range. In some embodiments, the supramolecular gels maintain their gel properties from temperatures ranging from 10° C. to 60° C. In preferred embodiments, the supramolecular gels maintain their gel properties at temperatures at or around room temperature (i.e. between about 20° C. and 22° C., e.g. about 21° C.). However, the gel compositions of the invention may be exposed to temperatures as low as 4° C. and temperatures as high as 60° C. and above, preferably from 4 to 25° C. The gel compositions of the invention will remain stable and maintain their structural integrity at such temperatures. An assessment of the stability of the supramolecular gels at particular temperatures (and, optionally, an assessment of their suitability to achieve the necessary requirements for storing and/or transporting and/or releasing of an encapsulated component at particular temperatures) may be performed using any of the methods described herein.

The skilled person will readily be able to further characterise a particular supramolecular gel to assess whether it possesses the necessary characteristics of a supramolecular gel of this invention. For example, the skilled person can assess or analyse:
the molecular interactions between gelator components (e.g. by NMR spectroscopy, IR spectroscopy, X-ray diffusion, UV-vis absorption or a combination thereof);
the fibre structures (e.g. by circular dichroism, UV-vis absorption spectroscopy, fluorescence spectroscopy, X-ray diffraction, IR spectroscopy, SANS/SAXS microscopy or a combination thereof;
the three-dimensional network structures (e.g. by scanning electron microscopy (SEM), (cryo-)transmission electron microscopy, confocal microscopy, etc.); and
the rheology of the gel (e.g. by using the techniques described herein).

As used herein, the term “component” or “encapsulated component” refers to any component (e.g. a (macro)molecule) that can be releasably-trapped within the three-dimensional network of the supramolecular gel of the invention. The “encapsulated component” of the present invention can be considered as a cargo that can be releasably-stored and transported within the structure of the gel.

As used herein, the term “releasably-stored” or “releasably-trapped” are used interchangeably herein to mean that an encapsulated component trapped within the structure of the supramolecular gels of the present invention can subsequently be released therefrom in response to a particular stimulus.

It will therefore be understood that such an encapsulated component can be stored and/or transported within the spaces between the networks of the supramolecular gel compositions of the invention.

The encapsulated component is not irreversibly trapped within the gel network; it can be released as described herein. The encapsulated component is one which is capable of passing through a filter having a 2.7 μm, 0.45 μm or 0.2 μm pore size. In some embodiments, the component may be a labile component, i.e. one which is unstable or aggregatable. In some embodiments, the encapsulated component is a water-soluble or water-dispersible moiety.

In some preferred embodiments, the encapsulated component comprises a biological macromolecule (e.g. a protein, polypeptide, peptide, hormone, nucleic acid (DNA or RNA), lipid or carbohydrate, etc.). The biological macromolecule may be naturally-occurring or synthetic. In some preferred embodiments, the encapsulated component comprises a therapeutic or a non-therapeutic agent, e.g. a protein. In some embodiments, the encapsulated component comprises s a pharmaceutical drug. For example, the encapsulated component may comprise a biopharmaceutical drug (e.g. a biologic). Thus, in some embodiments, the encapsulated component comprises a vaccine, an antibody (e.g. a monoclonal antibody), an enzyme, a blood factor, a thrombolytic agent, a hormone, a growth factor, an interferon or interleukin-based product.

In other embodiments, the encapsulated component is a formulation, e.g. a lipid nanoparticle (e.g. lipid-encapsulated mRNA). In some embodiments, the encapsulated component may further comprise a buffer, e.g. Tris, PBS, HEPES, acetate buffer. The encapsulated component may also comprise one or more sugars, salts, antibiotics, antivirals.

The encapsulated component is releasably trapped within the spaces formed in the gel network. Thus, in some embodiments, the encapsulated component is releasably trapped such that diffusion within the gel network is substantially or completely prevented. As such, the encapsulation of particular components described herein within the supramolecular gel will prevent or reduce aggregation of the encapsulated component.

In some embodiments, the encapsulated component is present in the supramolecular gel at a concentration of up to 10 wt %, e.g. 1-5 or 5-10%, for example, concentrations of about 1, 2, 3, 4, 5, 6, 7, 8, 9 or 10 wt %.

The invention also provides a process for preparing a supramolecular gel, the process comprising the steps:
(a) preparing a composition comprising one or more low molecular weight gelators (LMWGs) in an aqueous solution; and
(b) triggering the gelation of the LMWGs in the composition;
   under conditions that such that a supramolecular gel is formed from a network of the one or more LMWGs in the aqueous solution;
wherein the supramolecular gel:
(i) has a storage modulus (G′) of from 100 to 1,000,000 Pa;
(ii) has a loss modulus (G″) such that G″/G′ is from 0.01 to 0.5;
(iii) has a breakage strain of from 0.1 to 300%;
   and wherein:
(iv) at least 90% of the LMWGs in the supramolecular gel are capable of being removed by filtration of the gel composition through a filter.

The invention also encompasses supramolecular gels which are obtained or are obtainable by such a process.

The aqueous solution may, for example, be water (preferably deionised water) or an appropriate aqueous buffer. The aqueous solution may also comprise a component to be encapsulated.

As used herein, the term “gelation” refers to the process of forming a supramolecular gel from the one or more LMWGs in the aqueous solution.

The conditions which are required to produce a supramolecular gel having the properties specified herein from a given aqueous solution of one or more LMWGs will depend upon the specific LMWGs and the nature of the aqueous solution; but these conditions will be determinable by the person of skill in the art following the teachings herein (particularly the Examples herein) and this person's common general knowledge.

In particular, the disclosure herein provides the use of number of different LMWGs; references to a number of further potential gelators; and details of how to test the further LMWGs to see whether they are capable of forming supramolecular gels which have the desired properties. Furthermore, the triggering steps mentioned below may also be performed by the skilled person without an undue burden.

The formation of a supramolecular gel from LMWGs (such as those discussed herein) may be triggered by a number of means known in the art. The skilled person would appreciate that the means by which gelation is triggered will depend on the choice of the specific LMWG being used.

For example, one method of triggering gelation may simply involve heating and cooling an aqueous solution of the LMWG. In such embodiments, the heating of the aqueous solution results in the dissolution of the LWMG in the aqueous solution; and the subsequent cooling of this solution results in the LMWGs becoming a gel. For example, a solution of 4 mg/mL 1,3;2,4-dibenzylidene-D-sorbitol-p,p′-dihydrazide (DBS-hydrazide) in water may be heated to boiling point in a sealed tube and then allowed to cool, thus forming a gel.

In some embodiments, gelation may be triggered by changing the pH to which the LMWG is exposed. In such embodiments, the particular LMWG used will be soluble (or at least dispersible) at a particular pH, but will become insoluble upon changing the pH. Under appropriate conditions, gelation will occur. Examples of the use of pH to trigger gelation are given in the Examples attached herein.

In some embodiments, the process of gelation may require further components (in addition to the LMWG). For example, gelation may require addition of salts such as potassium carbonate and/or calcium chloride, and/or the additional of gelator buffers.

Examples of the use of calcium to trigger gelation are given in the Examples attached herein. (See also “Salt-induced Hydrogels from Functionalised-Dipeptides”, L. Chen, T. O. McDonald and D. J. Adams, RSC Advances, 2013, 3, 8714-8720.)

In some embodiments, gelation may be induced by addition of a buffer (e.g. Tris) to a concentrated solution of LMWGs at an appropriate pH (e.g. pH 8).

In some embodiments, a combination of the above-described methods are used in the process of gelation. In some embodiments, the gelation process is instantaneous. In other embodiments, the gelation process take place in 1 hour or less.

The preparation of the supramolecular gel may take place in any suitable vessel or container. In some preferred embodiments, the gelation step takes place in the barrel of a syringe or other hollow prism.

In another embodiment, the invention provides a process for encapsulating a component, in a gel, the process comprising the step (a) encapsulating the component in a supramolecular gel of the invention.

The process of encapsulating a particular component in the supramolecular gel may be performed by any means known to the skilled person in the art. For example, a solution in which the component (to be encapsulated) is dissolved may form part of the aqueous solution which forms (or formed) the supramolecular gel.

The supramolecular gel of the invention is capable of storing and/or transporting a component capable of being encapsulated in said gel (i.e. an “encapsulated component”). It will therefore be understood that the supramolecular gel of the invention is suitable for storing and/or transporting an encapsulated component.

In another embodiment, the invention provides a method of storing a component, the method comprising the step (a) storing a gel composition of the invention wherein the component is encapsulated within the supramolecular gel at a temperature above 4° C., preferably above 10° C., for a duration of at least 24 hours.

The invention also provides a method of transporting a component, the method comprising the step (a) transporting a gel composition of the invention wherein the component is encapsulated within the supramolecular gel from a first location to a second location.

In another embodiment, the invention provides a method for reducing or preventing the aggregation of an aggregatable component (preferably a protein) during storage or transport of the component the process comprising the step (a) encapsulating the component, in a supramolecular gel of the invention.

In some embodiments, the gel compositions of the invention comprising an encapsulated component can be stored and/or transported at temperatures ranging from 4° C. to 60° C. In preferred embodiments, the gel compositions comprising an encapsulated component are stored and/or transported at temperatures at or around room temperature (i.e. between about 20° C. and 22° C., e.g. about 21° C.). However, the gel compositions of the invention may be exposed to temperatures as low as 4° C. and temperatures as high as 60° C. and above. The gel compositions of the invention will remain stable and maintain their structural integrity at such temperatures. It will therefore be understood that the gel compositions of the invention can protect the encapsulated component from temperature fluctuations during storage and/or transport.

In some embodiments, the gel compositions comprising the encapsulated component do not require any refrigeration (i.e. at a temperature below 4° C.) during storage and/or transport. In some embodiments, the gel compositions comprising the encapsulated component are stored and/or are transported at a temperature above 5° C. (e.g. from 5 to 35° C.).

The duration of the transport or storage may be at least 1 day, e.g. 1-5, 5-10 or 10-25 days. The first and second locations may, for example, be at least 50 or 100 miles apart.

Upon appropriate strain/compression, the structural integrity of the network of one or more LMWGs will be compromised and the supramolecular gel will break into a plurality of parts or fragments. The parts or fragments may comprise small numbers of entangled or cross-linked LMWGs. Any entrapped component will thereby be released. Thus, the encapsulated components (optionally including any solvents and/or buffers) may be released from the supramolecular gels and thus separated from the supramolecular gels.

In particular, the invention also provides a process of extracting an encapsulated component from a gel composition which comprises the encapsulated component, the process comprising the steps (a) forcing a gel composition of the invention which comprises an encapsulated component through a filter having a pore size such that all or substantially all of the LMWGs are retained within the filter; and (b) collecting encapsulated component which passes through the filter.

The force is a force which is sufficient to propel the gel composition through the filter without causing damage to the filter. Thus the encapsulated component is mechanically-releasable from the supramolecular gel, i.e. the force may be a mechanical force. As used herein, the term “mechanically-releasable” refers to any means that can exert a force (e.g. a straining or compressing force) on the supramolecular gel to cause breakage of the network of LMWGs and release of the encapsulated component.

In some embodiments, the non-covalent interactions of the gel networks described herein are reversible when particular straining forces are applied to the supramolecular gel. In other words, the supramolecular gel structure is, in some embodiments, reversible under particular staining forces.

In such embodiments, when particular straining forces applied to the supramolecular gel, the gel network is reversed (i.e. the network is broken) and the gel components shatter into particles of a particular size.

The compression may be direct or indirect compression. For example, the compression may be a mechanical compression being applied to one or more faces of the supramolecular gel.

In some embodiments, prior to release of the encapsulated component, a stressor is applied to the gel composition of the invention in order to promote the release of the encapsulated component from the supramolecular gel. The stressor may, in some embodiments, be considered a form of agitation that prepares the supramolecular gel for breakage. In some embodiments, the stressor is heat, physical agitation or pH change.

In some preferred embodiments, the supramolecular gel has previously been formed and/or is present within the barrel of a syringe; and the mechanical compression is the direct action of the syringe plunger on one face of the supramolecular gel.

In other embodiments, the compression may be an indirect compression of the supramolecular gel, for example, the action of a centripetal force on the supramolecular gel whilst in a centrifuge. In such embodiments, the supramolecular gel would be present within a centrifuge tube comprising a filter.

The filter has a pore size such that at least 90% of the LMWGs in the supramolecular gel are capable of being removed by filtration of the gel composition through the filter. Preferably, at least 95% or at least 99% (by weight) of the LMWGs in the supramolecular gel will be removed by filtration. For example, in embodiments of the invention wherein the gel has previously been formed and/or is present within the barrel of a syringe, a syringe filter having the desired pore size may be connected to the syringe, and the syringe plunger may be used to compress the gel and to expel its contents though the filter. In the context of the invention, at least 90% of the LMWGs in the composition should be capable of being retained by the filter, i.e. less than 10% of the LMWGs in the gel composition should pass through the filter.

The filter will comprise pores, which can be of the same or different sizes. The filter has a pore size such that all or substantially all of the LMWGs are retained within the filter. The skilled person will readily be able to select an appropriate filter pore size to enable the encapsulated components to pass through the filter while preventing the passage of all or substantially all of the gel components through the filter.

The pore size of the filter is preferably from 0.1 μm to 3 μm, e.g. 0.1 to 0.5 μm, 0.5 to 1.0 μm or 1.0 to 3.0 μm. In some embodiments, the filter has a pore size of about 0.2 μm, 0.45 μm or 2.7 μm. In some embodiments, the filter is a paper filter.

In particular, at least 90% (preferably, at least 95% or at least 99%, by weight) of the LMWGs in the supramolecular gel will be retained by a filter having a 2.7 μm pore size, when the gel composition is forced through the filter.

Any encapsulated components will pass through the filter. It will be readily appreciated that other means to separate such encapsulated components could equally be used and which are known to the person skilled in the art. In particular, the invention is not limited to methods which comprise the expulsion of the encapsulated components through a filter having a 2.7 μm pore size.

As discussed herein, upon the application of an appropriate force, the network of the one or more LMWGs will break down, thus releasing any encapsulated component.

In one particularly-preferred embodiment, the extraction of the encapsulated component from a gel composition of the invention comprising the encapsulated component is performed using a syringe, wherein the gel composition is present in the barrel of the syringe. In such embodiments, the syringe will be equipped with a filter through which the encapsulated component may be forced while retaining all or substantially all of the LMWGs within the filter. Syringes are well-known in the art. Generally, a syringe comprises a hub portion (e.g. portion that connects a filter or needle portion to the barrel portion), a barrel portion (e.g. a cylindrical chamber that holds the contents to be pushed towards and through the hub portion) and a plunger portion (e.g. that which is mechanically operable to act upon the contents of the barrel portion to push the contents out of the syringe). Different types syringes are known in the art and any of these are suitable for use in methods of the present invention. For example, the syringe may be a glass syringe, a plastic syringe, a metal (e.g. a (stainless) steel) syringe or a combination of such materials.

In some preferred embodiments, the barrel portion of the syringe comprises a supramolecular gel composition of the invention, optionally additionally comprising an encapsulated component.

It will therefore be understood that, in such embodiments, the plunger portion of the syringe acts upon the gel composition within the barrel portion to exert a force (e.g. a straining or compressing force) upon the gel composition, thereby causing the gel formed from the network of one or more LMWGs to break down.

In some preferred embodiments, the syringe comprises a filter attached thereto, e.g. attached to the hub portion. Thus, in certain embodiments, the filter is arranged such that it filters the contents of the barrel portion as these contents are expelled from the barrel portion.

In a particularly-preferred embodiment, the invention provides a syringe having a barrel portion, wherein a gel composition of the invention is present within the barrel portion. In some embodiments, the syringe additionally comprises a hub portion and a plunger portion, wherein a filter is attached to the hub portion. The contents of the syringe's barrel portion may be expelled therefrom through the filter by the depression of the plunger portion.

In some embodiments, the released or collected encapsulated component is in a pure or substantially pure form. As used herein, the term “pure form” refers to the released or collected encapsulated component being substantially or completely free from any the agents or components which were used for gelation (e.g. LMWGs and excipients, etc.). The released or collected encapsulated component can therefore be considered homogenous. In some embodiments, the released or collected encapsulated component retains at least 80% or 90% of its functionality after storage in the supramolecular gel at 5° C. for 7 days (compared to its activity or functionality before encapsulation in the supramolecular gel). For example, in embodiments where the encapsulated component is a protein, the released or collected protein will retain at least 80%, preferably at least 90%, and most preferably 100% of its functional activity (compared to its activity prior to encapsulation in the supramolecular gel).

In some embodiments, the released or collected encapsulated component comprises a buffer. In some embodiments, the LMWGs may be separated from any expansion fluid (e.g. water and/or solvent and/or buffer) and, optionally, any encapsulated component therein by, for example, filtration.

In some embodiments, the encapsulated component is released in a biomedically-relevant volume. For example, the volume of released or collected encapsulated component may be 0.1 mL to 0.5 mL. In some embodiments, at least 50%, preferably at least 60% or at least 70% and most preferably at least 80%, of the encapsulated component originally present in the supramolecular gel composition is released or collected.

In another embodiment, the invention provides a kit comprising (i) one or more low molecular weight gelators (LMWGs); and (ii) a syringe, optionally together with instructions for use. The kit may also comprise a component, as defined herein.

### EMBODIMENTS

In certain aspects, the invention also provides the following Embodiments:
1. A gel composition comprising:
   (a) a supramolecular gel formed from or comprising a network of one or more low molecular weight gelators (LMWGs) in an aqueous solution;
wherein the supramolecular gel:
   (i) has a storage modulus (G′) of from 100 to 1,000,000 Pa;
   (ii) has a loss modulus (G″) such that G″/G′ is from 0.01 to 0.5; and
   (iii) has a breakage strain of from 0.1 to 300%.
2. The gel composition of Embodiment 1, wherein:
   (iv) at least a portion of the LMWGs in the supramolecular gel is capable of being removed by filtration of the gel composition through a filter.
3. The gel composition according to Embodiment 1 or Embodiment 2, wherein the LMWGs have a molecular weight of 100 to 3,000 Da.
4. The gel composition according to any one of Embodiments 1-3, wherein the storage modulus is 500 to 100,000 Pa.
5. The gel composition according to any one of Embodiments 1-4, wherein the loss modulus is 100 to 80,000 Pa.
6. The gel composition according to any one of Embodiments 1-5, wherein G″/G′ is 0.01 to 0.2.
7. The gel composition according to any one of Embodiments 1-6, wherein the breakage strain is 1 to 10%.
8. The gel composition according to any one of Embodiments 2-7, wherein the portion is at least 50% (or at least 55%, 60%, 65%, 70%, 75%, 80%, 85%, 90%, 95%, 96%, 97%, 98%, 99% or 100%) of the LMWGs in the supramolecular gel.
9. The gel composition according to any one of Embodiments 1-8, wherein the filter has a pore size of 0.1 μm to 3 μm.
10. The gel composition according to any one of Embodiments 1-9, which additionally comprises:
   (b) an encapsulated component, wherein the encapsulated component is encapsulated within the supramolecular gel.
11. The gel composition according to Embodiment 10, wherein the encapsulated component is a biological macromolecule.
12. The gel composition according to Embodiment 10 or 11, wherein the biological macromolecule is selected from the group consisting of a protein, polypeptide, peptide, hormone, nucleic acid, lipid and carbohydrate.
13. The gel composition according to any one of Embodiments 10-12, wherein the encapsulated component is a therapeutic or non-therapeutic agent.
14. A syringe comprising a gel composition according to any one of Embodiments 10-13, optionally wherein the syringe additionally comprises a filter and/or a needle.
15. A transdermal patch or a microneedle patch comprising a gel composition according to any one of Embodiments 10-13.
16. A flexible storage tube comprising a gel composition according to any one of Embodiments 10-13.
17. A process for preparing a supramolecular gel, the process comprising the steps:
   (a) preparing a composition comprising one or more low molecular weight gelators (LMWGs) in an aqueous solution; and
   (b) triggering the gelation of the LMWGs in the composition;
under conditions such that a supramolecular gel is formed from a network of the one or more LMWGs in the aqueous solution;
   wherein the supramolecular gel:
   (i) has a storage modulus (G′) of from 100 to 1,000,000 Pa;
   (ii) has a loss modulus (G″) such that G″/G′ is from 0.01 to 0.5;
   (iii) has a breakage strain of from 0.1 to 300%.
18. The process according to Embodiment 17, wherein:
   (iv) at least a portion of the LMWGs in the supramolecular gel is capable of being removed by filtration of the gel composition through a filter.
19. The process according to Embodiment 18, wherein the portion is at least 50% (or at least 55%, 60%, 65%, 70%, 75%, 80%, 85%, 90%, 95%, 96%, 97%, 98%, 99% or 100%) of the LMWGs in the supramolecular gel.
20. A process for encapsulating a component in a gel, the process comprising the step:
(a) encapsulating the component in a supramolecular gel as defined in any one of Embodiments 1-9.
21. The process according to Embodiment 20, wherein the component is an encapsulated component as defined in any one of Embodiments 10-13.
22. A method of storing a component, the method comprising the step:
(a) storing a gel composition as defined in any one of Embodiments 1-9, wherein the component is encapsulated within the supramolecular gel at a temperature above 4° C. for a duration of at least 24 hours.
23. The method according to Embodiment 22, wherein the component is an encapsulated component as defined in any one of Embodiments 10-13.
24. A method of transporting a component, the method comprising the step:
(a) transporting a gel composition as defined in any one of Embodiments 1-9, wherein the component is encapsulated within the supramolecular gel from a first location to a second location.
25. The method according to Embodiment 24, wherein the component is an encapsulated component as defined in any one of Embodiments 10-13.
26. A process of extracting an encapsulated component from a gel composition which comprises the encapsulated component, the process comprising the steps:
(a) forcing a gel composition as defined in any one of Embodiments 1-9 which comprises the encapsulated component through a filter having a pore size such that at least a portion of the LMWGs are retained within the filter; and
(b) collecting encapsulated component which passes through the filter.
27. The process according to Embodiment 26, wherein the encapsulated component is as defined in any one of Embodiments 10-13.
28. The process according to Embodiment 26 or Embodiment 27, wherein the portion is at least 50% (or at least 55%, 60%, 65%, 70%, 75%, 80%, 85%, 90%, 95%, 96%, 97%, 98%, 99% or 100%) of the LMWGs.
29. A kit comprising:
(i) one or more low molecular weight gelators (LMWGs); and
(ii) a syringe,
optionally together with instructions for use.

In some Embodiments, at least 50% (preferably at least 55%, 60%, 65%, 70%, 75%, 80%, 85%, 90%, 95% or 100% by weight) of the LMWGs in the supramolecular gel will be retained by a filter having a 0.2 μm pore size, when the gel composition is forced through the filter.

In some Embodiments, at least 50% (preferably at least 55%, 60%, 65%, 70%, 75%, 80%, 85%, 90%, 95% or 100% by weight) of the LMWGs in the supramolecular gel will be retained by a filter having a 0.45 μm pore size, when the gel composition is forced through the filter.

In some Embodiments, at least 50% (preferably at least 55%, 60%, 65%, 70%, 75%, 80%, 85%, 90%, 95% or 100% by weight) of the LMWGs in the supramolecular gel will be retained by a filter having a 2.7 μm pore size, when the gel composition is forced through the filter.

In some Embodiments, the supramolecular gel comprises aqueous CaCl₂, preferably at a concentration of 1.0-80.0 mM, more preferably 5.0-20.0 mM and most preferably at a concentration of about 7.1 mM.

In some Embodiments, the supramolecular gel comprises a buffer. Any suitable buffer (e.g. a physiologically-acceptable buffer) may be used including, but not limited to, TRIS-HCl, HEPES, PBS or acetate buffer preferably at a concentration of 0.1-2.0 M, more preferably at a concentration of about 1.0-1.5M, most preferably about 1.1 M. The buffer is preferably at pH 6.6-7.0, more preferably about pH 6.8.

In some Embodiments, the supramolecular gel comprises a Tris-HCl buffer preferably at a concentration of 0.1-2.0 M, more preferably at a concentration of about 1.0-1.5M, and most preferably about 1.1 M. The Tris-HCl buffer is preferably at pH 6.6-7.0, and more preferably about pH 6.8.

The encapsulated component may be present in the syringe, patch or tube in an effective amount or effective concentration.

As used herein, the term “effective amount” or “effective concentration” refers to the amount the encapsulated component that is sufficient to achieve a beneficial or desired result (e.g. therapeutically or cosmetically).

Examples of therapeutic agents include, but are not limited to, vitamins, metabolites, molecular or chemical probes, steroids or corticosteroids, neurotransmitters, receptor antagonists, GPCR ligands, ion channel modulators, signalling activators and inhibitors, anticancer drugs, non-steroidal anti-inflammatory drugs (NSAIDS), antibiotics, an anti-inflammatory (e.g., Diclofenac), a pain reliever (e.g., Bupivacaine), a Calcium channel blocker (e.g., Nifedipine), an Antibiotic (e.g., Ciprofloxacin), a Cell cycle inhibitor (e.g., Simvastatin), chemotherapeutics, anti-viral drugs, antineoplastic agents, local anaesthetics, angiogenic agents, anti-angiogenic agents, growth factors, psychoactive drugs, drugs affecting reproductive organs, genes, and oligonucleotides, or other targets.

Examples of non-therapeutic agents include cosmetic agents.

In accordance with another aspect, the invention further provides a transdermal patch or a microneedle patch comprising a gel composition according to the invention.

Microneedle patches comprise an array of microneedles which permit delivery of the encapsulated component through the skin of the subject (i.e. a wearer of such a microneedle patch).

Preferably, the subject is a human subject.

In some embodiments, a transdermal patch or a microneedle patch according to the present invention comprises a filter as described herein.

In accordance with another aspect, the invention further provides a flexible storage tube comprising a gel composition according to the invention.

As used herein, the term “flexible storage tube” refers to a squeezable tube that is resistant to moisture and air, and is capable of storing a gel composition of the invention (preferably along with an encapsulated component as described herein). Such flexible storage tubes of the invention may also protect the contents of the storage tube from light.

In some Embodiments, the flexible storage tube of the invention comprises a filter as described herein.

In all embodiments of the present invention comprising a filter, it will be readily understood that the filter may be positioned such that, in the process of extracting an encapsulated component, the gel composition of the invention (comprising an encapsulated component) is forced through the filter. For example, the filter may be in-line with a syringe or flexible storage tube comprising a gel composition of the invention.

The disclosure of each reference set forth herein is specifically incorporated herein by reference in its entirety.

### BRIEF DESCRIPTION OF THE FIGURES

FIG. 1 shows the initial gel studies. (A) Chemical structure of the gelator used here. (B) Photographs of example gels formed (left) at pH 6.8 and (right) at pH 6.8 in the presence of a calcium salt. (C) Typical strain sweep for gels formed in the absence and presence of a calcium salt, showing a high stiffness (G′) but a low breakage strain. Filled squares represent G′ of gels formed in the presence of calcium salt; filled triangles represent G″ of gels formed in the presence of calcium salt; open circles represent G′ of gels formed in the absence of calcium salt; and open triangles represent G″ of gels formed in the absence of calcium salt. (D) SAXS patterns of gels made in presence of (top) calcium chloride and (bottom) without CaCl₂. The fits obtained through model fitting are over-layed on each spectrum). (E) CD spectra of gels in presence of (solid line) calcium chloride and (dotted line) without CaCl₂. (F) Cartoon showing the concept of this work. (G) Images exemplifying the syringe filter release protocol for gels. The gel was first loaded in a syringe fitted with a 2.7 μm filter. The gel was passed through the filter by gentle extrusion, releasing a clear solution.

FIG. 2 shows the preliminary gel studies. (A) CD-005 gels formed using CaCl₂ encapsulating dextran (left, 1 wt %; right, 10 wt %). (B) Rheological data for gels formed encapsulating dextran. Measurements were performed in triplicate and plotted data are presented as mean+/−s.d. Filled circles=G′. Open circles=G″. (C) Rheological data for gels formed encapsulating 1 wt % dextran over time. Measurements were performed in triplicate and plotted data are presented as mean+/−s.d. Filled circles=G′. Open circles=G″. (D) Extrusion through a syringe filter releases the entrapped material. (E) NMR evidence showing that no gelator was released during extrusion. The peaks at around 5.0 and 7.6 ppm are from the buffer.

FIG. 3 shows retention of protein function in the CD-005 gels of the invention. (A) General preparation process for gel; (B) Probe for aggregation inhibition using Insulin with THT assay; (C) Quantification of THT assay; (D) β-galactosidase assay for post-recovery function; (E) Activity recovery after 7 days at 50° C. for independent repeats; F) Comparison of 1 week and 4 week storage and recovery of β-galactosidase.

FIG. 4 shows exemplary gelator structures of use in the invention. For (a), gelation was achieved by adding a concentration solution at pH 8 to a buffer. For (b)-(f), gels were formed by a pH switch from a solution at pH 10.5 giving gels at a final pH 4.

FIG. 5 shows insulin aggregation assay using dynamic light scattering with alternative gelators (2NapIF and 1 NapFF). Gels were incubated at 25° C. for 24 hours at 900 rpm.

FIG. 6 shows the recovery of beta-galactoside from CD-005 gels following return postage. (A) Raw data of ONPG hydrolysis by beta-galactosidase; (B) Activity recovery as a percentage of fresh.

### EXAMPLES

The present invention is further illustrated by the following Examples, in which parts and percentages are by weight and degrees are Celsius, unless otherwise stated. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. Thus, various modifications of the invention in addition to those shown and described herein will be apparent to those skilled in the art from the foregoing description. Such modifications are also intended to fall within the scope of the appended claims.

### Materials and Methods

The following Materials and Methods were used in one or more of the Examples.

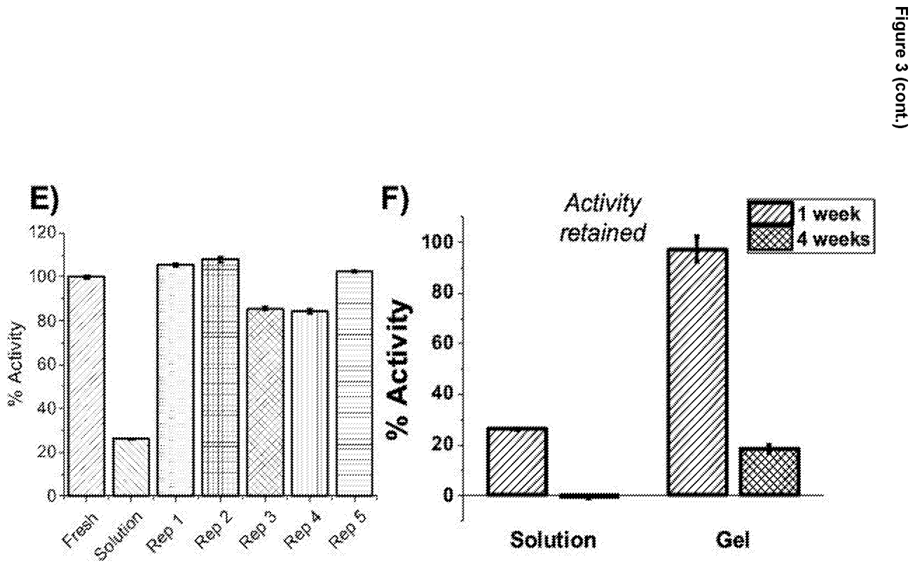

All chemicals were purchased from Sigma-Aldrich and used as received. Deionized water was used throughout. The gelator used here was synthesised following the protocols of Angulo-Pachón and Miravet.²⁵ Full details are described below. The other gelators tested (FIG. 4) were prepared as described elsewhere.²⁶

pH measurements. The pH of solutions and gels was measured using a FC200 pH probe (Hanna instruments) calibrated using pH 4.01, 7.01 and 10.01 buffer solutions. The probe was rinsed with deionised water between measurements.

Rheology. Rheological measurements were carried out using Anton Paar Physica MCR301 and M101 Rheometers. A cup and vane (ST10-4V-8.8/97.5-SN42404) system was used for all frequency and strain sweeps, with a measuring gap of 1.35 mm. Gels were prepared directly in 7 mL Sterilin vials, which were loaded on to the rheometer and measured in situ to ensure that no damage was carried out to the gels by transfer from vials. Strain sweeps were performed from 0.08% to 1000% at a frequency of 10 rad/s. Frequency sweeps were performed from 1 rad/s to 100 rad/s at a constant strain of 0.5% (within the linear viscoelastic region for all gels).

Small Angle X-ray scattering. Small angle X-ray scattering experiments were performed at Diamond Light Source, Didcot at the 122 beamline. The beamline operates at an energy of 12.5 keV and the camera length was set to 4.275 m to give a Q range of 0.002-0.30 Å. The gels were prepared as described in the next section and immediately loaded in glass capillaries using a 1 mL syringe with a 21 G needle. The raw data was processed using the DAWN Science software (version 2.27),²⁷ according to a standard 122 pipeline.²⁸ As part of the processing, the backgrounds were subtracted from the raw 2D SAXS data and a full azimuthal integration was performed to reduce the data to a I vs q plot. The plots were then fitted to structural models using the SasView software (version 5.0.4).

Formation of gels for CD-005. CD-005 gels were prepared either with or without the addition of CaCl₂. For both, 50 mg of CD-005 were added to 20 mg of K₂CO₃ and dissolved in 5 mL de-ionised water under overnight stirring. For the samples without CaCl₂, 0.4 mL of the gelator solution were transferred to a vial, to which 1 mL of 1.5M Tris HCl (pH 6.8) was added. For the samples formed with the calcium trigger, 5.5 μL of a 200 mg/mL CaCl₂ solution were first added to 1 mL of 1.5M Tris HCl (pH 6.8) in a separate vial. This was then added in one aliquot to 0.4 mL of gelator buffer. For rheological measurements, the gels were prepared in vials and left undisturbed overnight on the bench. To prepare the samples in syringes, the gels were prepared as above and immediately transferred (<1 minute) to a syringe through a 21 G needle. It is relevant to note that all the samples needed to be prepared at 19° C. to ensure optimal gelation.

Preparation of other tested gels by reduction in pH using GdL. Micellar solutions were prepared as previously described at concentrations of 5 mg/mL.²⁶ Gel samples were prepared in 7 mL Sterilin vials by addition of 2 mL of stock solution (adjusted to pH 10.5) to 16 mg of solid GdL for 5 mg/mL solutions. The vials were swirled briefly by hand to ensure complete dissolution of GdL then left to stand overnight undisturbed.

Encapsulation of dextran, insulin and beta galactosidase. For the CD-005 gels, dextran was encapsulated in the gel by dissolution of dextran powder in 1.5M Tris HCl, pH 6.8 at a range of concentrations (0.14 wt % to 14 wt %). 1 mL of this solution was transferred to 0.4 mL of the gelator solution described above to achieve the desired final concentrations of dextran (0.1 wt % to 10 wt %). For the gels obtained by reduction in pH, the dextran was dissolved at the various required concentrations in de-ionised H₂O under stirring. These solutions were then used to prepare the micellar solutions of the gelators at 5 mg/mL

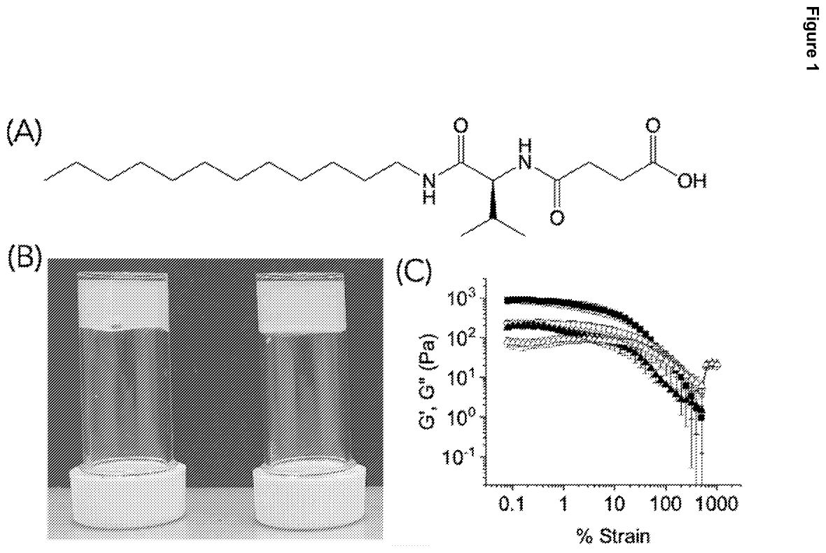

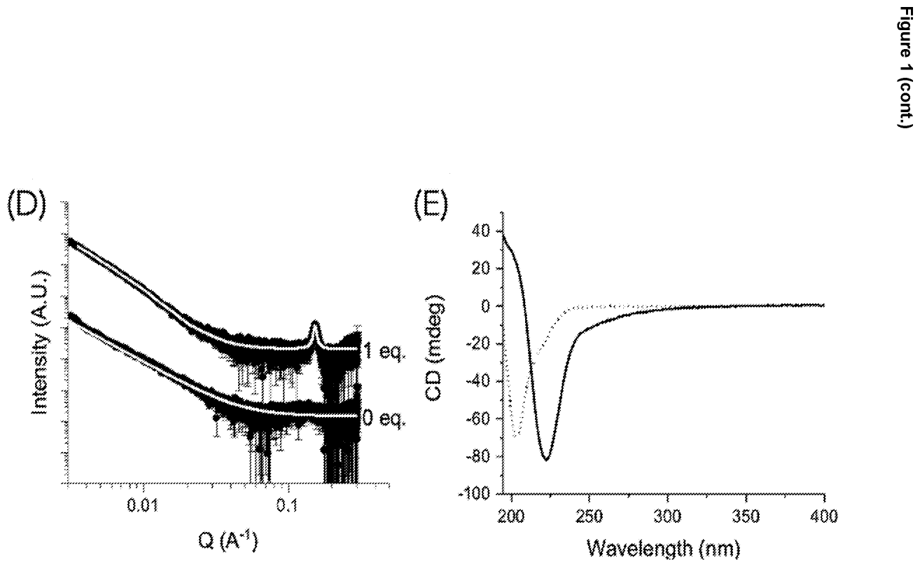

Release protocols through syringe filter. To release the gel with dextran inclusion, the gel prepared in the syringe was passed through a 2.7 μm filter. FIG. 1G exemplifies the methodology of release through the filter: the sample is firstly allowed to gel overnight in a syringe, then the gel is gently passed through the 2.7 μm syringe filter releasing a clear solution. Generally, ca. 80% of the liquid is recovered from the procedure.

Circular Dichroism. Circular dichroism data was acquired on a Chirascan VX spectrometer (Applied Photophysics) using a quart cuvette with a 0.01 mm path length. The spectra were collected in the range 180-400 nm with a scanning step size of 1.0 nm and scanning rate of 0.25 s at room temperature. The samples were prepared in Sterilin vials as described keeping the same volumes of the components. Small amount of the gels were then transferred to the cuvette prior to measurement.

FTIR spectroscopy. Data were recorded using an Agilent Cary 630 FTIR spectrometer (with ATR attachment). The filter paper from the 2.7 μm filter was removed by carefully opening the syringe filters. Then, the background of the empty ATR crystal was taken. Small amounts of a clean filter and one after extrusion were deposited on the ATR crystal to record the spectra.

### Insulin-Loaded Gel Preparations.

2NapIF. First, 1.1 ml of 0.1 M NaOH was added to 8.9 ml of dH₂O. 50 mg of 2NapIF was then added to this solution and left to dissolve overnight on a rotary shaker. Prior to gelation, insulin was added to a final concentration of 0.2 mg/ml. Gelation was initiated by adding 80 mg of GdL to this solution. The sample was then pipetted into 5×2 ml syringes before being sealed with parafilm and left overnight.

1 NapFF. First, 1 ml of 0.1 M NaOH was added to 9 ml of dH₂O. 50 mg of 1 NapFF was then added to this solution and left to dissolve overnight on a rotary shaker. Prior to gelation, insulin was added to a final concentration of 0.2 mg/ml. Gelation was initiated by adding 20 mg of GdL to this solution. The sample was then pipetted into 5×2 ml syringes before being sealed with parafilm and left overnight.

CD-005. 20 mg of potassium carbonate was dissolved in 5 ml of dH₂O. 50 mg of CD-005 was added to this solution and left to dissolve overnight on a rotary shaker. 4 ml of this solution was added to 10 ml of 1.5 M Tris-HCl at pH 6.8. Immediately after, insulin was added to a final concentration of 0.2 mg/ml. The sample was then pipetted into 5×2 ml syringes before being sealed with parafilm and left overnight.

Insulin storage and assay. Insulin in solution, along with insulin in gel (in syringe) were agitated on an Eppendorf Smartblock at 600 rpm, 25° C. for 6 hours. Prior to analysis, the samples in syringes were passed through a 0.22 um filter to separate protein from the gelators and then used in the assays.

Dynamic light scattering (DLS) was used to measure the hydrodynamic radius on a Zetasizer ZS (Malvern Panalytical, Worcestershire, UK). Measurements were carried out using a 4 mW He—Ne 633 nm laser module operating at 25° C. at an angle of 173° (back scattering), and results were analyzed using Malvern DTS 7.03 software. There were 10 replicated for each of the samples with at least 12 measurements recorded for each run.

ThT assay. 1 mM stock solution of Thioflavin T (ThT) is prepared in H₂O. This ThT was diluted in PBS (pH 7.4) so that the final ThT concentration in each well is 25 μM in 100 μl. Another 100 μl of insulin solution from each of the gelators after passing through 0.22 μm filter was added to the wells. ThT fluorescence was measured using a fluorescence microplate reader (excitation 450 nm, emission 485 nm).

Beta-Galactosidase Storage and Assay. Stock solutions of the gelator and protein were freshly prepared before mixing. 20 mg of KCO₃ was dissolved in 5 mL of distilled water, to which 50 mg of CD-005 (gelator) was added. This mixture was stirred overnight to dissolution. In a separate vial 5.5 μL of a 200 mg/mL CaCl₂ solution was added to 1 mL of 1.5M Tris HCl, pH 6.8. β-Galactosidase was added to give a 10 mg/mL solution. 0.4 mL of the gelator in buffer was transfer to a vial, to which 1 mL of the protein solution was added. These were agitated and rapidly (<1 minute) transferred into the syringe. Gel-loaded syringes were stored in a thermostated incubator for the indicated amount of time, before being retrieved and assayed for function. Protein was recovered from the syringe by passing through a syringe filter (0.22 μm). 50 μL of 100 μg/mL β-Galactosidase solution (this was adjusted using PBS as TRIS can inhibit the activity of this protein) was added into the wells of a 96 well plate, containing 100 μL of 16 mM ONPG (4.82 mg/mL). The absorbance was then measured at 420 nm, each minute for 10 minutes.

Postage Protocols. Gels containing beta-galactosidase were prepared as described above. Five gels and one solution of beta-galactosidase (i.e., in the absence of gel) were triple bagged and put in a parcel. They were posted using Royal Mail signed delivery. A temperature logger was included in the parcel. In total the package was in delivery for 3 days which showed a temperature range of 17.6 to 24.2° C. Upon receipt, samples were visually inspected for leaks (none were seen) and to confirm the gels were intact. Samples were recovered and assayed as above.

### Example 1: Formation and Testing of Gels Formed from LMWGs

We formed gels from a range of LMWGs (FIG. 4). Based on gelation at physiological conditions, we focused on one system (FIG. 1A). The advantage of this system is that gelation can be induced by the addition of a TRIS buffer solution to a concentrated solution of the LMWG at pH 8 to give a final self-supporting material at pH 6.8. The mechanical properties can be tuned by the addition of a calcium salt (FIG. 1B). In the absence of the calcium salt the system has relatively low storage (G′) and loss (G″) moduli, with the material breaking at low strain (<1%). In the presence of the calcium salt, the moduli are improved, but crucially the material still breaks at low strain (FIG. 1C). Using small angle X-ray scattering (SAXS) to probe the sample without salt, the scattering data can be fit simply to a power law (FIG. 1D, bottom), suggesting the presence of large heterogeneities in the network or irregular aggregates in the gel. The data for the gel prepared in presence of CaCl₂ could be fit to an elliptical cylinder model combined with a power law (FIG. 1D, top). The data further presents a peak at q=0.154 Å (d=40.8 Å), which suggests that the gelator fibers are part of a periodic arrangement in the structure with a crystalline character. Circular dichroism (CD) indicates a different arrangement in the secondary structures of the systems. The CD spectrum of the sample without any salt exhibits a negative band with a shoulder in the range of 200-240 nm (FIG. 1E, dotted line). The sample with CaCl₂ shows a negative band around 235 nm and a positive band occurring near 190 nm, which could be indicative of a β-sheet-like arrangement (FIG. 1E, solid line).

Encapsulating additives including proteins in gels formed by LMWG has previously been done.^{20,21} The proteins are trapped in the space between the fibers; if the gel network is sufficiently permanent and the pores between fibers sufficiently small, the protein is unable to easily diffuse within the gel and hence aggregate.²² The potential for these gels to break at low strain means we can form gels within a syringe for long term room temperature storage, followed by release through a syringe filter; the essentially insoluble extended supramolecular network will be trapped within the syringe filter, meaning that only the pure protein is released (FIGS. 1F and 1G).

### Example 2: Loading of LMWG Gels with Encapsulated Components

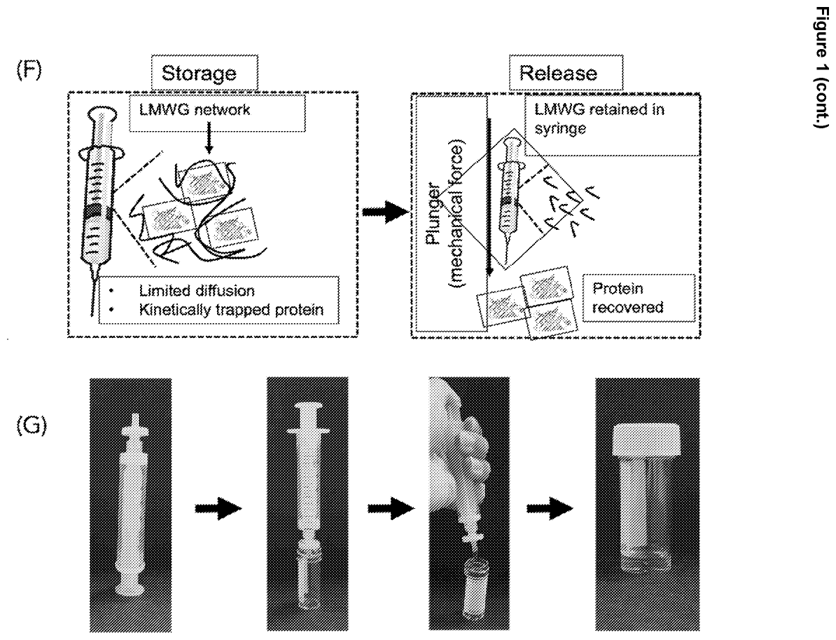

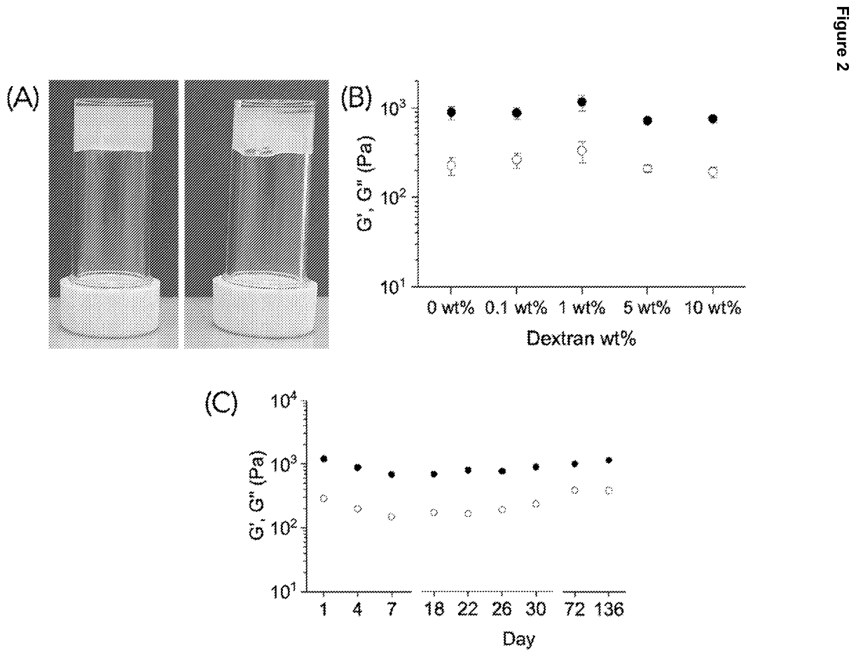

A model macromolecular cargo was loaded into CD-005 gels to probe the impact on gel structure, function, and release. Dextran (Mᵣ 6000 Da, hydrodynamic radius=1.6 nm²³) was chosen as a non-interacting additive. Dextran was added at concentrations up to 10 wt % (FIG. 2A, a value far above the loadings required for real-world drug storage and in line with recent moves towards higher concentration biologic formulations), whilst keeping all other parameters constant. Our gelation approach is almost instant; in comparison, chemically-triggered hydrogel storage solutions require addition of dosed release agents, such as sugars, and require up to an hour to gel.¹⁵ The gel properties were retained over all loadings, which is essential for a broadly applicable storage solution (FIG. 2B). Extended aging experiments showed that these properties did not change significantly over at least 136 days (FIG. 2C). The gels can be formed in situ inside a syringe. As initial proof-of-concept, we entrapped a fluorescent dextran within a gel and then extruded it through a syringe filter. Pure dextran was collected directly (FIG. 2D). No gelator could be detected in the extruded liquid by 1H NMR spectroscopy (FIG. 2E), showing that the gel network is trapped within the syringe filter and our simple approach leads to excipient-free homogeneous protein on demand. The gelator was trapped in the filter. To add further quantification, we used a LMWG which incorporated an inherently-fluorescent group. After extrusion, no fluorescence could be detected in the extrudates, showing the power of this approach

### Example 3: Protection from Aggregation of Insulin Provided by LMWG Gels

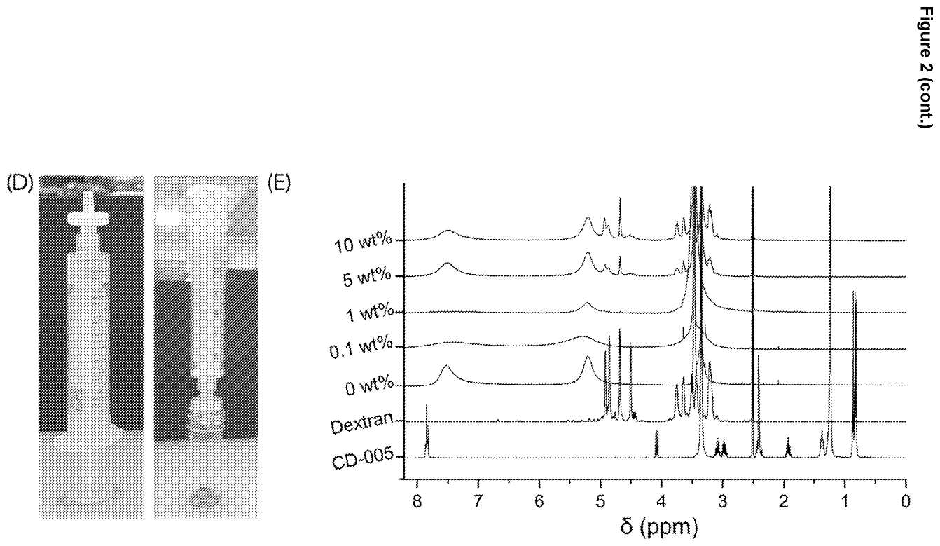

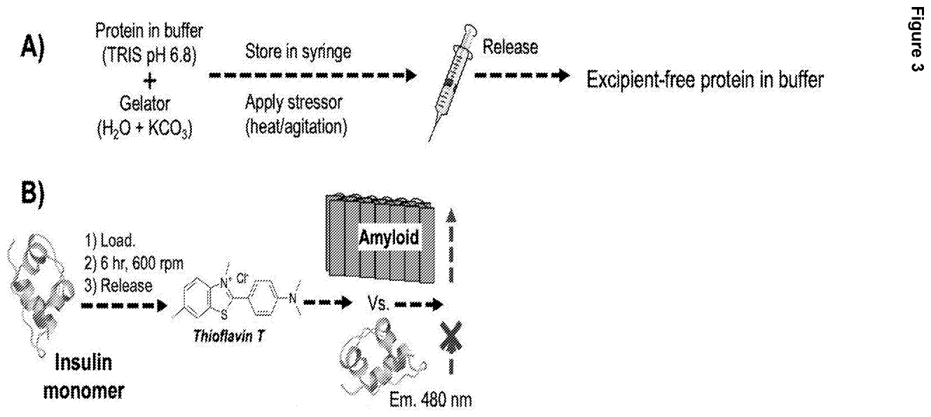

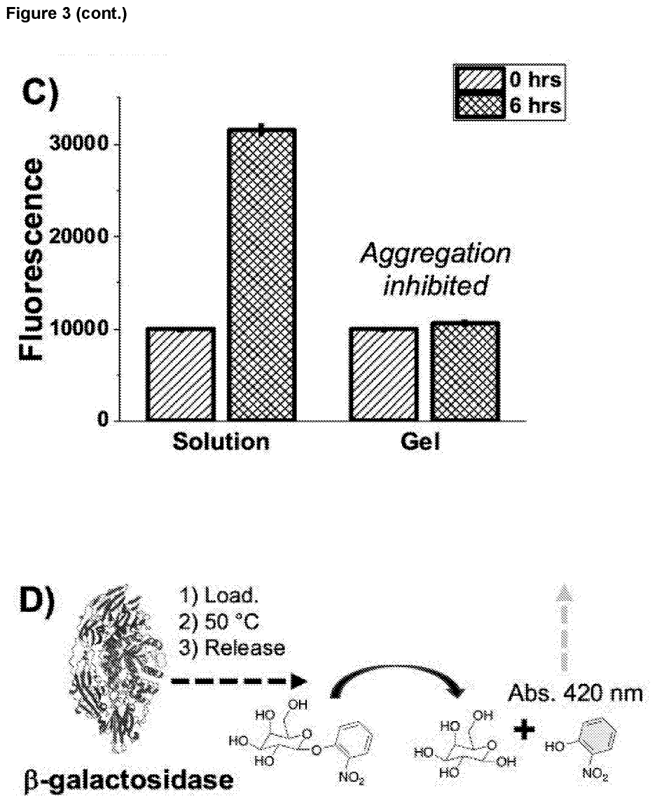

With confirmation that macromolecules can be encapsulated and selectively released by a mechanical trigger, protein storage was undertaken to demonstrate recovery and the mode of action (preventing aggregation). As a first challenge, we chose to encapsulate (bovine) insulin as a model therapeutic protein (FIG. 3A). Insulin is one of the most widely used protein drugs in the world but must be stored cool or freeze-dried requiring the user to self-prepare the solution prior to injection.²⁴ The instructions for insulin are very specific not to shake, as it is prone to aggregate into extended amyloid fibers losing efficacy and bioavailability.

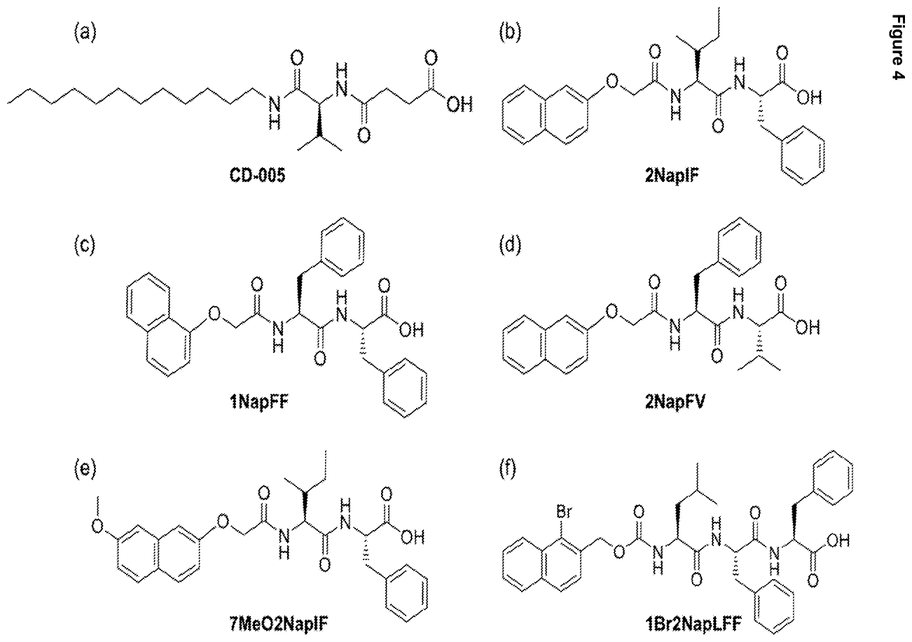

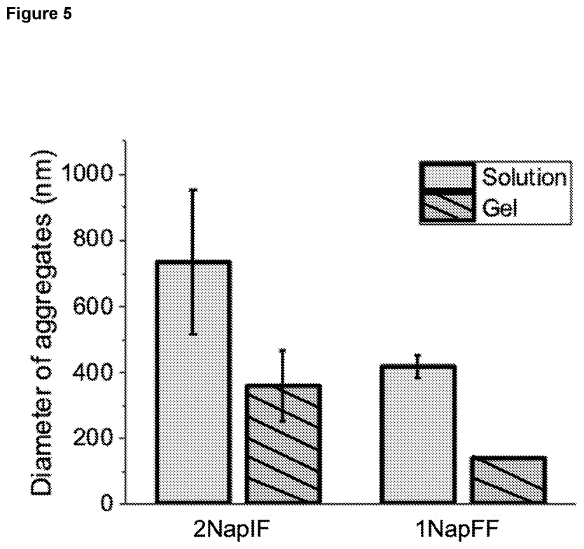

After initial screening using dynamic light scattering, insulin was loaded into CD-005 gels, which were then incubated at 25° C. and shaken at 600 rpm: this is very aggressive agitation far beyond a real-world stress. After this time, pure excipient-free protein was recovered by action of the syringe and any aggregation to amyloid was determined by the ThT (thioflavin T) assay (FIG. 3A). It should be noted, all samples were treated identically, and the solution-only samples were passed through the syringe filter, so there was no bias due to filtered/exclusion of aggregates. Limited aggregation was detected in the gel loaded samples, but extensive aggregation occurred in the absence of the gel. In no case was gelator detected in the solutions extruded through the syringe filter. Hence, our approach provides dual protection against aggregation and allows protein release in a pure form. Insulin aggregation was also assessed in solution versus gel form using dynamic light scattering with two other gelators (FIG. 5). Aggregation was again limited in the gel as compared to the samples with insulin in solution.

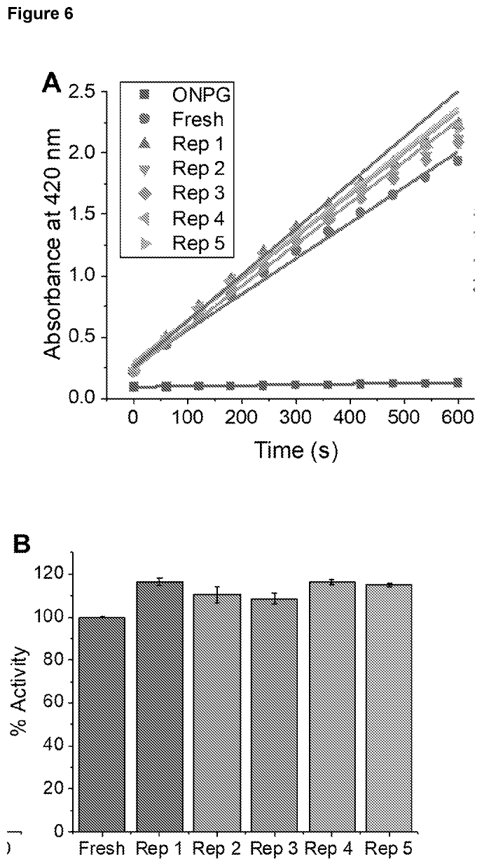

Insulin provided an aggregative readout, but it is also important to ensure protein function could be retained. Therefore, beta galactosidase (β-gal) was encapsulated. When folded correctly, β-gal catalyses the decomposition of p-nitrophenyl-galactoside allowing colorimetric evaluation of activity. The CD-005 gels were placed in an incubator at 50° C. (a very stringent test beyond feasible transport conditions) for 4 days. After this time, protein was recovered by simple syringe action. Compared to fresh protein, β-gal in buffer alone retained just 26% of its function, but across 15 repeats (3 technical, 5 biological) the gel-stored protein recovered 84-100% of function with a mean of 97.1±2.8%. This is equal performance to a novel multi-armed boronic-acid function PEG hydrogel,¹⁰ but our technology functions at biomedically relevant volumes (0.5 mL verses 50 μL), does not require addition of any triggers such as sugars or acid, and the resulting protein was excipient free. As an even more challenging test, gels were stored at 50° C. but for 4 weeks. This removed all activity from the solution-stored β-gal, but the gel-stored protein retained up to 20% of their function: this is a remarkable level of recovery from a harsh storage condition at conditions where even the hottest locations on earth rarely face. To show the gels are robust, samples were mailed through the UK postal service to experience a realistic distribution challenge (shaking, dropping, and exposure to a range of temperatures). Over the 2-day delivery period, the gels were recovered intact and ~100% activity was retained (FIG. 6), showing that this method could conceptually be used for the postal delivery of active proteins for healthcare.

### Example 4: Filtration of Gels Through Filters with Different Pore Sizes

CD-005 gels were prepared either with or without the addition of CaCl₂ according to the “Formation of gels for CD-005” section, discussed above. The gels were immediately transferred to a syringe after their preparation. The gels were then forced out of the syringes through filters of varying pore sizes and the % of solid gelator filtered from the gels was determined.

The results are shown below in the following table:

| | | % of CD-005 coming |
|---|---|---|
| Gel | Pore Size (μm) | through the filter |
| Tris-HCl | 0.2 | 4.57% |
| | 0.45 | 2.85% |
| | 2.7 | 61.75% |
| | No syringe filter | 90.75% |
| Tris-HCl + CaCl₂ | 0.2 | 0% |
| | 0.45 | 0% |
| | 2.7 | 0% |
| | No syringe filter | 100% |

The results show that for the gel without CaCl₂ the 0.2 μm and 0.45 μm filter pore sizes resulted in <5% of the CD-005 gelator passing through the filter and out of the syringe in each case. For the gel with CaCl₂ present, none of the CD-005 gelator was observed to pass through the filter and out of the syringe in each case.

### CONCLUSIONS

It has been demonstrated herein that the function of encapsulated components (e.g. proteins) is retained in stiff low molecular weight gels when stored at temperatures as high as 50° C. for up to 4 weeks by preventing irreversible aggregation. Due to the unique mechanical properties of the low molecular weight gels of the invention, they break when passed through an in-line syringe filter releasing the fully functional (protein) cargo with no need for any chemical triggers; and the effect is instant. Due to the extended supramolecular network formed by the gelators of the invention, a portion or all of the gel components (e.g., excipients) are retained in the filter, meaning essentially only the encapsulated component (e.g. protein) and buffer are released. To be very clear, this means only pure encapsulated component (e.g. protein) in buffer is delivered, unlike all competing technologies which lead to the encapsulated component (e.g. protein) containing novel, often untested, excipients which would then be delivered to a patient, and hence face regulatory and safety barriers to use. This is also a benefit over freezer-dried or frozen formulations requiring no use of reconstituted buffer, or energy-intensive ultra-low freezers, respectively. We have therefore provided a new scalable means of storing, transporting, and safely using biological agents and non-therapeutic agents affording a simple and effective strategy to deliver biologic therapies and non-therapeutic agents to low-resource settings where they are needed most.

### REFERENCES

1 Walsh, G. Biopharmaceutical benchmarks 2014. Nature Biotechnology 32, 992-1000, doi:10.1038/nbt.3040 (2014).
2 Hafer, M. Quantity and electricity consumption of plug load equipment on a university campus. Energy Efficiency 10, 1013-1039, doi:10.1007/s12053-016-9503-2 (2017).
3 Kaushik, J. K. & Bhat, R. Why Is Trehalose an Exceptional Protein Stabilizer?: AN ANALYSIS OF THE THERMAL STABILITY OF PROTEINS IN THE PRESENCE OF THE COMPATIBLE OSMOLYTE TREHALOSE*. Journal of Biological Chemistry 278, 26458-26465, doi:https://doi.org/10.1074/jbc.M300815200 (2003).
4 Anchordoquy, T. J. & Carpenter, J. F. Polymers Protect Lactate Dehydrogenase during Freeze-Drying by Inhibiting Dissociation in the Frozen State. Archives of Biochemistry and Biophysics 332, 231-238, doi:https://doi.org/10.1006/abbi.1996.0337 (1996).
5 Jain, N. K. et al. Formulation and stabilization of recombinant protein based virus-like particle vaccines. Advanced Drug Delivery Reviews 93, 42-55, doi:https://doi.org/10.1016/j.addr.2014.10.023 (2015).
6 Crommelin, D. J. A., Anchordoquy, T. J., Volkin, D. B., Jiskoot, W. & Mastrobattista, E. Addressing the Cold Reality of mRNA Vaccine Stability. Journal of Pharmaceutical Sciences 110, 997-1001, doi:10.1016/j.xphs.2020.12.006 (2021).
7 Kumru, O. S. et al. Vaccine instability in the cold chain: Mechanisms, analysis and formulation strategies. Biologicals 42, 237-259, doi:https://doi.org/10.1016/j.biologicals.2014.05.007 (2014).
8 van Beers, M. M. C., Jiskoot, W. & Schellekens, H. On the Role of Aggregates in the Immunogenicity of Recombinant Human Interferon Beta in Patients with Multiple Sclerosis. Journal of Interferon & Cytokine Research 30, 767-775, doi:10.1089/jir.2010.0086 (2010).
9 Lee, J. et al. Trehalose Glycopolymers as Excipients for Protein Stabilization. Biomacromolecules 14, 2561-2569, doi:10.1021/bm4003046 (2013).
10 Marco-Dufort, B. et al. Thermal stabilization of diverse biologics using reversible hydrogels. Science Advances 8, eabo0502, doi:10.1126/sciadv.abo0502.
11 Sridhar, B. V. et al. Thermal Stabilization of Biologics with Photoresponsive Hydrogels. Biomacromolecules 19, 740-747, doi:10.1021/acs.biomac.7b01507 (2018).
12 vander Straeten, A. et al. A microneedle vaccine printer for thermostable COVID-19 mRNA vaccines. Nature Biotechnology, doi:10.1038/s41587-023-01774-z (2023).
13 Georgiou, P. G., Kontopoulou, I., Congdon, T. R. & Gibson, M. I. Ice recrystallisation inhibiting polymer nano-objects via saline-tolerant polymerisation-induced self-assembly. Materials Horizons 7, 1883-1887, doi:10.1039/DOMH00354A (2020).
14 Chen, B.-M., Cheng, T.-L. & Roffler, S. R. Polyethylene Glycol Immunogenicity: Theoretical, Clinical, and Practical Aspects of Anti-Polyethylene Glycol Antibodies. ACS Nano 15, 14022-14048, doi:10.1021/acsnano.1c05922 (2021).
15 Marco-Dufort, B. et al. Thermal stabilization of diverse biologics using reversible hydrogels. Science Advances 8, eabo0502, doi:doi:10.1126/sciadv.abo0502 (2022).
16 Terech, P. & Weiss, R. G. Low Molecular Mass Gelators of Organic Liquids and the Properties of Their Gels. Chemical Reviews 97, 3133-3160, doi:10.1021/cr9700282 (1997).
17 Draper, E. R. & Adams, D. J. Low-Molecular-Weight Gels: The State of the Art. Chem 3, 390-410, doi:https://doi.org/10.1016/j.chempr.2017.07.012 (2017).
18 Piras, C. C., Genever, P. G. & Smith, D. K. Combining gellan gum with a functional low-molecular-weight gelator to assemble stiff shaped hybrid hydrogels for stem cell growth. Materials Advances 3, 7966-7975, doi:10.1039/D2MA00565D (2022).
19 Wang, J. et al. Incorporation of supramolecular hydrogels into agarose hydrogels—a potential drug delivery carrier. Journal of Materials Chemistry 19, 7892-7896, doi:10.1039/B913158B (2009).
20 Altunbas, A., Lee, S. J., Rajasekaran, S. A., Schneider, J. P. & Pochan, D. J. Encapsulation of curcumin in self-assembling peptide hydrogels as injectable drug delivery vehicles. Biomaterials 32, 5906-5914, doi:https://doi.org/10.1016/j.biomaterials.2011.04.069 (2011).
21 Raymond, D. M. et al. Low-Molecular-Weight Supramolecular Hydrogels for Sustained and Localized in Vivo Drug Delivery. ACS Applied Bio Materials 2, 2116-2124, doi:10.1021/acsabm.9b00125 (2019).
22 Faidra Angelerou, M. G. et al. Mechanistic investigations into the encapsulation and release of small molecules and proteins from a supramolecular nucleoside gel in vitro and in vivo. Journal of Controlled Release 317, 118-129, doi:https://doi.org/10.1016/j.jconrel.2019.10.011 (2020).
23 Wallace, M., Adams, D. J. & Iggo, J. A. Analysis of the mesh size in a supramolecular hydrogel by PFG-NMR spectroscopy. Soft Matter 9, 5483-5491, doi:10.1039/C3SM27793C (2013).
24 Qafary, M., Rashno, F., Khajeh, K., Khaledi, M. & Moosavi-Movahedi, A. A. Insulin fibrillation: Strategies for inhibition. Progress in Biophysics and Molecular Biology 175, 49-62, doi:https://doi.org/10.1016/j.pbiomolbio.2022.09.001 (2022).
25 Angulo-Pachón, C. A. & Miravet, J. F. Sucrose-fueled, energy dissipative, transient formation of molecular hydrogels mediated by yeast activity. Chemical Communications 52, 5398-5401, doi:10.1039/C6CC01183G (2016).
26 Gupta, J. K., Adams, D. J. & Berry, N. G. Will it gel? Successful computational prediction of peptide gelators using physicochemical properties and molecular fingerprints. Chemical Science 7, 4713-4719, doi:10.1039/C6SC00722H (2016).
27 Filik, J. et al. Processing two-dimensional X-ray diffraction and small-angle scattering data in DAWN 2. Journal of Applied Crystallography 50, 959-966, doi:doi:10.1107/S1600576717004708 (2017).
28 Pauw, B. R., Smith, A. J., Snow, T., Terrill, N. J. & Thunemann, A. F. The modular small-angle X-ray scattering data correction sequence. Journal of Applied Crystallography 50, 1800-1811, doi:doi:10.1107/S1600576717015096 (2017).

1. A gel composition comprising:
(a) a supramolecular gel formed from or comprising a network of one or more low molecular weight gelators (LMWGs) in an aqueous solution;
wherein the supramolecular gel:
(i) has a storage modulus (G′) of from 100 to 1,000,000 Pa;
(ii) has a loss modulus (G″) such that G″/G′ is from 0.01 to 0.5; and
(iii) has a breakage strain of from 0.1 to 300%;
and wherein:
(iv) at least 90% of the LMWGs in the supramolecular gel are capable of being removed by filtration of the gel composition through a filter.

2. The gel composition as claimed in claim 1, wherein the LMWGs have a molecular weight of 100 to 3,000 Da.

3. The gel composition as claimed in claim 1 or claim 2, wherein the storage modulus is 500 to 100,000 Pa.

4. The gel composition as claimed in any one of the preceding claims, wherein the loss modulus is 100 to 80,000 Pa.

5. The gel composition as claimed in any one of the preceding claims, wherein G″/G′ is 0.01 to 0.2.

6. The gel composition as claimed in any one of the preceding claims, wherein the breakage strain is 1 to 10%.

7. The gel composition as claimed in any one of the preceding claims, wherein the filter has a pore size of 0.1 μm to 3 μm.

8. The gel composition as claimed in any one of the preceding claims, which additionally comprises:
(b) an encapsulated component, wherein the encapsulated component is encapsulated within the supramolecular gel.

9. The gel composition as claimed in claim 8, wherein the encapsulated component is a biological macromolecule, preferably selected from the group consisting of a protein, polypeptide, peptide, hormone, nucleic acid, lipid and carbohydrate.

10. A syringe comprising a gel composition as claimed in claim 8 or claim 9.

11. A process for preparing a supramolecular gel, the process comprising the steps:
(a) preparing a composition comprising one or more low molecular weight gelators (LMWGs) in an aqueous solution; and
(b) triggering the gelation of the LMWGs in the composition;
under conditions that such that a supramolecular gel is formed from a network of the one or more LMWGs in the aqueous solution:
wherein the supramolecular gel:
(i) has a storage modulus (G′) of from 100 to 1,000,000 Pa;
(ii) has a loss modulus (G″) such that G″/G′ is from 0.01 to 0.5;
(iii) has a breakage strain of from 0.1 to 300%;
and wherein:
(iv) at least 90% of the LMWGs in the supramolecular gel are capable of being removed by filtration of the gel composition through a filter.

12. A process for encapsulating a component in a gel, the process comprising the step:
(a) encapsulating the component in a supramolecular gel as defined in any one of claims 1 to 7, preferably wherein the component is an encapsulated component as defined in claim 9.

13. A method of storing a component, the method comprising the step:
(a) storing a gel composition as defined in any one of claims 1 to 7 wherein the component is encapsulated within the supramolecular gel at a temperature above 4° C. for a duration of at least 24 hours, preferably wherein the component is as defined in claim 9.

14. A method of transporting a component, the method comprising the step:
(a) transporting a gel composition as defined in any one of claims 1 to 7 wherein the component is encapsulated within the supramolecular gel from a first location to a second location, preferably wherein the component is an encapsulated component as defined in claim 9.

15. A process of extracting an encapsulated component from a gel composition which comprises the encapsulated component, the process comprising the steps:
(a) forcing a gel composition as defined in any one of claims 1 to 7 which comprises the encapsulated component through a filter having a pore size such that all or substantially all of the LMWGs are retained within the filter; and
(b) collecting encapsulated component which passes through the filter, preferably wherein the component is an encapsulated component as defined in claim 9.

16. A kit comprising:
(i) one or more low molecular weight gelators (LMWGs); and
(ii) a syringe,
optionally together with instructions for use.
